(19) United States
(12) Patent Application Publication
(10) Pub. No.: US 2025/0235577 A1
(43) Pub. Date: Jul. 24, 2025

(54) DISINFECTION AND IN SITU FLOCCULATION-SOLIDIFICATION PROCESS FOR PATHOGENIC MEDICAL WASTE DISPOSAL

(71) Applicant: COUNCIL OF SCIENTIFIC AND INDUSTRIAL RESEARCH AN INDIAN REGISTERED BODY INCORPORATED UNDER THE REGN., New Delhi, Delhi (IN)

(72) Inventors: Sreejith Shankar POOPPANAL, Kerala Thiruvananthapuram (IN); Sruthi Surendran NAIR, Kerala Thiruvananthapuram (IN); Achu RADHAKRISHNAKURUP, Kerala Thiruvananthapuram (IN); Visakh VIJAYAN, Kerala Thiruvananthapuram (IN); Peer Mohamed Abdul AZEEZ, Kerala Thiruvananthapuram (IN); Hareesh Unnikrishnan Nair SARASWATHY, Kerala Thiruvananthapuram (IN); Rajeev Kumar SUKUMARAN, Kerala Thiruvananthapuram (IN); Savithri SIVARAMAN, Kerala Thiruvananthapuram (IN); Parukkuttyamma Devi SUJATHA, Kerala Thiruvananthapuram (IN); Ajayaghosh AYYAPPANPILLAI, Kerala Thiruvananthapuram (IN)

(73) Assignee: COUNCIL OF SCIENTIFIC AND INDUSTRIAL RESEARCH AN INDIAN REGISTERED BODY INCORPORATED UNDER THE REGN., New Delhi, Delhi (IN)

(21) Appl. No.: 18/698,473

(22) PCT Filed: Sep. 22, 2022
(86) PCT No.: PCT/IN2022/050847

§ 371 Date: Apr. 4, 2024

(30) Foreign Application Priority Data
Oct. 4, 2021 (IN) 202111045341

(51) Int. Cl. A61L 11/00; A01N 59/00; A01P 1/00; A61L 2/18; A61L 2/23; A61L 101/12; A61L 101/24; A61L 101/30; A61L 101/36

(52) U.S. Cl. CPC A61L 11/00; A01N 59/00; A01P 1/00; A61L 2/18; A61L 2/23; A61L 2101/12; A61L 2101/24; A61L 2101/30; A61L 2101/36; A61L 2202/121; A61L 2202/122; A61L 2202/22

(57) ABSTRACT

The present invention intends to disclose a process for in situ flocculation followed by solidification of biomedical waste that is capable of simultaneously treating and disinfecting solid and fluid samples. The process comprises of the addition of the waste samples to an alkaline aqueous solution of metal silicates followed by the addition of an organic or inorganic acid for flocculation and a solid metal oxide or phosphate at a defined volumetric and/or weighted composition leading to instantaneous solidification with >99.9% microbial disinfection and an all-in-one disinfecting device for treatment of biomedical waste. The present disclosure also provides a disinfection-flocculation-solidification and disposal device comprising the disinfection composition.

### FIELD OF THE INVENTION

The present invention relates to new process for the efficient flocculation and/or solidification of biomedical waste that is capable of simultaneously treating and disinfecting solid and fluid samples. Particularly, the present invention relates to the process for disinfecting biomedical wastes comprising of the addition of the waste samples to an alkaline aqueous solution of metal silicates followed by the addition of an inorganic or organic acid leading to a flocculated state and further addition of a metal oxide or phosphate based solid powders at a defined volumetric and/or weighted composition leading to instantaneous solidification with >99.9% microbial disinfection. More particularly, the present invention relates to a disinfecting device for treatment of biomedical waste.

### BACKGROUND AND PRIOR ART OF THE INVENTION

Mismanagement of infectious wastes such as biomedical test samples leads to the transmission of microbes/toxins/viruses and further steer the spread of contagious and infectious diseases. According to a position statement (2000) by WHO, improper management of medical wastes such as infected hypodermic needles and syringes has caused infections pertaining to hepatitis B (21 million cases), hepatitis C (2 million cases) and HIV (0.26 million cases) worldwide. The following statements quoted from WHO undermine the significance and need for proper medical waste management: “Poor management of medical waste potentially exposes healthcare workers, waste handlers, patients, and the community at large to infection, toxic effects and injuries, and risks polluting the environment. It is essential that all medical waste materials are segregated at the point of generation, appropriately treated, and disposed of safely” (Reproduced from http://www.who.int/topics/medical_waste/en/).

Adding a flocculating agent to liquid waste reduces the risk of spills and aerosolization. Solid wastes such as cotton, sharps as well as tissue papers may also lead to spread of infections and simple absorbers or hypochlorites that are currently in use are not always capable of treating such wastes. If the flocculating/gelling agent contains a disinfectant, it may be possible to dispose of the waste as non-regulated medical waste, which is less expensive than red-bagging. Segregation, transportation, and incineration of such disinfected medical wastes are easier, safer and decrease medical waste disposal costs for a healthcare facility.

Several strategies have been adopted for the management of liquid biomedical waste and include, but not limited to, sanitary sewer disposal methods, chemical treatment using 1% sodium hypochlorite solution with a minimum contact period of 30 min or 10-14 g bleaching powder per liter of water, 70% ethanol, 4% formaldehyde, 70% isopropyl alcohol, 25% iodine or 6% hydrogen peroxide, solidification of liquid waste using dry super adsorbent polymers containing sanitizers or disinfecting agents like chlorine or glutaraldehyde, closed disposal systems, etc. Reference may be made to an article, “Liquid biomedical waste management: An emerging concern for physicians, Biswal S, Muller J Med Sci Res 2013, 4, 99-106” which states that the culture media containing high microbial loads or rich protein contents requires rigorous disinfection procedures, wherein inactivation is achieved using 5.23% sodium hypochlorite in a 1:10 dilution for a minimum of 8 h inside a secured vessel followed by disposal down the sanitary sewer and subsequent flushing with a lot of cold water for at least of 10 min.

Solidification systems (super adsorbents) are deemed advantageous over other methods for the treatment and safer disposal of biomedical fluid wastes. Superabsorbent polymers are generally prepared polymerizing unsaturated carboxylic acids or derivatives thereof, including, but not limited to, acrylic acid or its or metal/ammonium salts and alkyl acrylates, using an internal cross-linking agent such as oligo-functional monomers including, but not limited to, bisacrylamides, triacrylates, dimethacrylates, or triallylamines.

Several patents have educated the development of such solidification systems. Application Reference may be made to the U.S. Pat. No. 7,291,674B2 wherein surface cross-linked superabsorbent polymers with good liquid retention, permeability, and mechanical strength based on the absorbent structure.

Reference may be made to the U.S. Pat. No. 8,450,389B1, wherein one or a plurality of surface cross-linked superabsorbent particles in combination with a plurality of second particles for liquid solidification with reduced gel block and a method of solidifying liquid medical waste

Reference may be made to another patent U.S. Pat. No. 9,533,081B1, wherein a portable wound therapy system comprising a plurality of surface cross-linked superabsorbent particles along with a container, a wound covering, and a packet and included a similar liquid solidification system with reduced gel block.

Reference may be made to the U.S. Pat. No. 5,391,351A, wherein a body waste fluid solidification device comprising a hydrophilic xerogel of partially hydrolyzed poly(vinyl acetate), cross-linked poly(vinyl alcohol), cross-linked hydroxyalkyl acrylates and methacrylates, polymers and copolymers of ethylene oxide and polymers and copolymers acrylamide

Reference may be made to the U.S. Pat. No. 6,797,857B2, wherein a solidifier for the solidification of a volume of liquid with a known density, comprising of three adsorbents with varying densities, thereby achieving controlled stabilization of a flowable material throughout its overall volume.

Reference may be made to the U.S. Pat. No. 5,424,265A, wherein a capsule for absorbing liquid waste with a powder adsorbent material disposed within said capsule, the body of the said capsule being water soluble leads to the adsorption of liquid waste located within a suction canister.

Reference may be made to the U.S. Pat. No. 9,102,806B2, wherein a particulate superabsorbent polymer capable of absorbing water, aqueous liquids, and blood, and a process to manufacture the said superabsorbent polymers. The said super adsorbent comprises of a 1-10 wt % of a thermoplastic polymer of any class selected from polyolefin, polyethylene, linear low-density polyethylene, ethylene acrylic acid copolymer, styrene copolymers, ethylene alkyl methacrylate copolymer, polypropylene, ethylene vinyl acetate copolymer, polyamide, polyester, blends thereof, or copolymers thereof, where the surface is treated with a neutralized multivalent metal salt solution having a pH value similar to that of human skin.

Reference may be made to the U.S. Pat. No. 8,403,904B2, wherein a superabsorbent polymer comprising an internal cross-linking agent consisting of a silane derivative having a minimum of one vinyl group or one allyl group attached to a silicon atom, and at least one Si—O bond with high centrifuge retention capacity.

Super adsorbing polymers, methods for their preparation and application in liquid solidification have been described by several patents, viz, EP2739660B2, US20130310251A1, EP0273141B1, U.S. Pat. No. 8,476,189B1, JP5527916B2, U.S. Pat. No. 5,578,318A, DE69815670T2, U.S. Pat. No. 8,821,363B1.

Solid wastes including, but not limited to, used cotton, tissue papers, syringes and needles are generally disinfected using approved disinfectants and/or sanitizers and are incinerated or recycled. Waste burial or land-fills, disposal in cemented pits, immobilization using plastic foam, sand, cement or clay, low/medium/high temperature burning, controlled incineration, steam autoclaving, rotary kiln, microwave treatment, chemical treatment, shredding, melting, etc. are the general practices in disposing solid waste (Reference may be made to WHO @ www.who.int/, and Medical Waste Management, International Committee of the Red Cross @ www.icrc.org/). A 1-10% solution of bleach, or hypochlorites, sodium hydroxide or other chemical disinfectants are used to disinfect biomedical waste. Heat, alkaline digesters, and microwaves are also used for this purpose.

Acrylate based solidifiers, though cheap and vastly available, are not devoid of disadvantages. It generally takes 10-15 min for complete gelation and are not easily recycled. They are non-biodegradable, and some acrylates are shown to be flammable. Studies have indicated that several acrylates and their raw materials can be carcinogenic. Manufacturing of acrylics has both health and environmental impacts. Several chemicals used in the manufacturing as well as the chemical waste from acrylic plants are toxic. Hypochlorite (bleach) is not always effective with high organic content waste such as blood. Further, a disinfection system capable of instantaneously treating, immobilizing, and disinfecting both liquid and solid medical wastes is not found in literature.

### ABBREVIATIONS USED

WHO: World Health Organization
min.: minutes
wt %: weight percentage
NaOH: Sodium hydroxide
mg: milligram
mL: milliliter
kg: kilogram OBJECTIVES OF THE INVENTION

The primary objective of the present invention relates to the development of an efficient flocculation or solidification system that is capable of disinfecting solid and fluid biomedical waste samples.

Another objective is to provide a simple and cost-effective process for the preparation for disposal of solid and fluid waste at the required point of care either in its flocculated or solidified forms.

A third objective is to provide an easy, safe, and cost-effective strategy for reducing the risks of spillage and occupational exposure from handling biomedical waste samples.

Yet another objective is to develop a process for the preparation for disposal of solid and fluid biomedical waste by destroying or disinfecting or deactivating infectious agents such as bacteria, virus, etc. via in situ flocculation followed by solidification as required.

### SUMMARY OF THE INVENTION

The present invention intends to disclose a process for the disinfection and flocculation or solidification of biomedical waste. The process involves the use of alkaline solution of metal silicates, organic or inorganic acids as flocculating agent and solid powders of a solidifying agent, which when subjected to mixing with solid or fluid waste samples at a defined volumetric and/or weighted composition leads to instantaneous flocculation or solidification with up to 100% microbial disinfection.

The present invention discloses a process for disinfection followed by in situ flocculation and solidification by disinfection-flocculation-solidification and disposal system, wherein disinfection composition comprising of a four chemical components A, B, C and D, wherein: a) A is a silicate salt of an alkali metal selected from the group consisting of sodium, potassium, and combinations thereof in its 20-40% aqueous solution at a concentration of 0.5-80% (w/v); b) B is a base at a concentration of 0.1-90% w/v added to an aqueous solution of A; c) C is an organic or inorganic acid miscible completely in water; and d) D is a solidifying agent selected from an oxide or a phosphate based powder inter alia oxides/phosphates of silicon, titanium, zinc, aluminum or lanthanide elements such as cerium or lanthanum.

The present invention intends to provide a disinfection process for the preparation for disposal of solid and fluid wastes collected in a collection vessel combined with the destruction, disinfection or deactivation of infectious agents including microorganisms inter alia bacteria, fungus etc., viruses and other toxins, whereby the disposal including treatment, handling and transportation are deemed easier, safer and cost-effective.

The present invention provides a process for disinfection-flocculation-solidification of wastes, comprising the steps of: a) addition of B to an aqueous solution of A; b) addition of a biomedical waste to be disinfected to the said aqueous solution as prepared in step (a); c) homogeneous mixing of the mixture as in (b) and/or resting for 10-30 min, wherein the obtained mixture is characterized as flocculated; and d) addition of material C and material D as its solid powder followed by mixing and/or resting, wherein the obtained mixture is characterized as solidified.

Another object of the present invention provides a method to create a flocculated or non-pourable environment for fluid medical wastes inter alia salt, sugar, saliva, urine, blood, hospital chemicals, etc. wherein risks related to spillage and occupational exposure are minimized, and further to the treatment of solid medical wastes inter alia cotton, tissue paper, swabs, needles, etc., wherein the risks related to accumulation of untreated and infected samples are minimized or a mixture of solid and liquid wastes added with >99.9% microbial disinfection.

In another embodiment, the present invention discloses the process involving an aqueous solution of a metal silicate containing a pH regulating base or alkali for complete disinfection of fluid or solid medical waste followed by the addition of an organic or inorganic acid for flocculation or any oxide based solid powder, as a single or plurality of the said powders, for instantaneous solidification of solid or fluid samples containing proteins, microbial cultures, salt or metal ions in high concentrations.

In a final object, the invention intends to create all-in-one sample collection-disinfection-solidification devices of requisite dimensions capable of collecting the solid or liquid sample, and immobilizing them as and when required with prior pathogenic disinfection for preparation for its disposal.

These and other features, aspects, and advantages of the present subject matter will become better understood with reference to the following description. This summary is provided to introduce a selection of concepts in a simplified form. This summary is not intended to identify key features or essential features of the disclosure, nor is it intended to be used to limit the scope of the subject matter.

### BRIEF DESCRIPTION OF THE FIGURES

FIG. 1 illustrates the flocculation process involving saturated salt (NaCl) solution upon addition of acetic acid: (a) 1 mL 28% sodium silicate solution (aq.) containing 0.3 g NaOH, (b) 1 mL saturated salt solution, (c) 1 mL 50% aqueous NaOH+1 mL saturated salt solution and (d) after addition of acetic acid for flocculation, in accordance with an embodiment of the present disclosure.

FIG. 2 illustrates the solidification process involving saturated salt (NaCl) solution upon addition of acetic acid and silica gel (chromatographic grade, 60-120 mesh): (a) 1 mL 28% sodium silicate solution (aq.) containing 0.3 g NaOH, (b) 1 mL saturated salt solution, (c) 1 mL 50% aqueous NaOH+1 mL saturated salt solution and (d) after addition of acetic acid for flocculation followed by silica gel for solidification, in accordance with an embodiment of the present disclosure.

FIG. 3 illustrates the solidification process involving saturated sugar (sucrose) solution upon addition of acetic acid and silica gel (chromatographic grade, 60-120 mesh): (a) 1 mL 28% sodium silicate solution (aq.) containing 0.3 g NaOH, (b) 1 mL saturated sugar solution, (c) 1 mL 50% aqueous NaOH+1 mL saturated sugar solution and (d) after addition of acetic acid for flocculation followed by silica gel for solidification, in accordance with an embodiment of the present disclosure.

FIG. 4 illustrates the solidification process involving a mixture of saturated salt (NaCl) and sugar (sucrose) solutions upon addition of acetic acid and silica gel (chromatographic grade, 60-120 mesh): (a) 1 mL 28% sodium silicate solution (aq.) containing 0.3 g NaOH, (b) 0.5 mL saturated salt+0.5 mL saturated sugar solutions, (c) 1 mL 50% aqueous NaOH+1 mL saturated salt+sugar solutions and (d) after addition of acetic acid for flocculation followed by silica gel for solidification, in accordance with an embodiment of the present disclosure.

FIG. 5 illustrates the solidification process involving aqueous waste solutions upon addition of hydrochloric acid and silica gel (chromatographic grade, 60-120 mesh): (a) 1 mL 28% sodium silicate solution (aq.) containing 0.3 g NaOH, (b) 1 mL aqueous waste, (c) 1 mL 50% aqueous NaOH+1 mL aqueous waste and (d) after addition of hydrochloric acid for flocculation followed by silica gel for solidification, in accordance with an embodiment of the present disclosure.

FIG. 6 illustrates the solidification process involving 6% BSA solution upon addition of acetic acid and silica gel (chromatographic grade, 60-120 mesh): (a) 1 mL 28% sodium silicate solution (aq.) containing 0.3 g NaOH, (b) 6% BSA solution, (c) 1 mL 50% aqueous NaOH+1 mL 6% BSA solution and (d) after addition of acetic acid for flocculation followed by silica gel for solidification, in accordance with an embodiment of the present disclosure.

FIG. 7 illustrates the solidification process involving a mixture of saturated salt (NaCl) and 6% BSA solutions upon addition of acetic acid and silica gel (chromatographic grade, 60-120 mesh): (a) 1 mL 28% sodium silicate solution (aq.) containing 0.3 g NaOH, (b) 0.5 mL saturated salt+0.5 mL 6% BSA solutions, (c) 1 mL 50% aqueous NaOH+1 mL saturated salt+6% BSA solutions and (d) after addition of acetic acid for flocculation followed by silica gel for solidification, in accordance with an embodiment of the present disclosure.

FIG. 8 illustrates the solidification process involving a mixture of saturated salt (NaCl) and 6% BSA solutions upon addition of acetic acid and silica gel (chromatographic grade, 60-120 mesh): (a) 1 mL 28% sodium silicate solution (aq.) containing 0.3 g NaOH, (b) 0.5 mL saturated salt+0.5 mL 6% BSA solutions, (c) 1 mL 50% aqueous NaOH+1 mL saturated salt+6% BSA solutions and (d) after addition of acetic acid for flocculation followed by silica gel for solidification, in accordance with an embodiment of the present disclosure.

FIG. 9 illustrates the flocculation process involving saturated potassium dichromate solution upon addition of acetic acid: (a) 1 mL 28% sodium silicate solution (aq.) containing 0.3 g NaOH, (b) 1 mL saturated potassium dichromate solution, (c) 1 mL 50% aqueous NaOH+1 mL saturated potassium dichromate solution and (d) after addition of acetic acid for flocculation, in accordance with an embodiment of the present disclosure.

FIG. 10 illustrates the solidification process involving saturated potassium dichromate solution upon addition of acetic acid and silica gel (chromatographic grade, 60-120 mesh): (a) 1 mL 28% sodium silicate solution (aq.) containing 0.3 g NaOH, (b) 1 mL saturated potassium dichromate solution, (c) 1 mL 50% aqueous NaOH+1 mL saturated potassium dichromate solution and (d) after addition of acetic acid for flocculation followed by silica gel for solidification, in accordance with an embodiment of the present disclosure.

FIG. 11 illustrates the solidification process involving iodine solution upon addition of acetic acid and silica gel (chromatographic grade, 60-120 mesh): (a) 1 mL 28% sodium silicate solution (aq.) containing 0.3 g NaOH, (b) 1 mL iodine solution, (c) 1 mL 50% aqueous NaOH+1 mL iodine solution and (d) after addition of acetic acid for flocculation followed by silica gel for solidification, in accordance with an embodiment of the present disclosure.

FIG. 12 illustrates the solidification process involving artificial blood upon addition of acetic acid and silica gel (chromatographic grade, 60-120 mesh): (a) 1 mL 28% sodium silicate solution (aq.) containing 0.3 g NaOH, (b) 1 mL artificial blood, (c) 1 mL 50% aqueous NaOH+1 mL artificial blood and (d) after addition of acetic acid for flocculation followed by silica gel for solidification. 6% BSA provided the protein content and heme was substituted with an iron(II) complex, in accordance with an embodiment of the present disclosure.

FIG. 13 illustrates the solidification process involving artificial urine upon addition of acetic acid and silica gel (chromatographic grade, 100-200 mesh): (a) 0.5 mL 28% sodium silicate solution (aq.) containing 0.15 g NaOH, (b) 0.5 mL 50% aqueous NaOH+0.5 mL artificial urine and (c) after addition of acetic acid for flocculation followed by silica gel for solidification, in accordance with an embodiment of the present disclosure.

FIG. 14 illustrates the solidification process involving artificial saliva upon addition of acetic acid and silica gel (chromatographic grade, 100-200 mesh): (a) 1 mL 28% sodium silicate solution (aq.) containing 0.3 g NaOH, (b) 1 mL 50% aqueous NaOH+1 mL artificial saliva and (c) after addition of acetic acid for flocculation followed by silica gel for solidification, in accordance with an embodiment of the present disclosure.

FIG. 15 illustrates the solidification process involving saturated potassium dichromate solution upon addition of acetic acid and silica gel (chromatographic grade, 100-200 mesh): (a) 1 mL 28% sodium silicate solution (aq.) containing 0.3 g NaOH, (b) 1 mL saturated potassium dichromate solution, (c) 1 mL 50% aqueous NaOH+1 mL saturated potassium dichromate solution and (d) after addition of acetic acid for flocculation followed by silica gel for solidification, in accordance with an embodiment of the present disclosure.

FIG. 16 illustrates the solidification process involving saturated potassium dichromate solution upon addition of acetic acid and silica gel (chromatographic grade, 230-400 mesh): (a) 1 mL 28% sodium silicate solution (aq.) containing 0.3 g NaOH, (b) 1 mL saturated potassium dichromate solution, (c) 1 mL 50% aqueous NaOH+1 mL saturated potassium dichromate solution and (d) after addition of acetic acid for flocculation followed by silica gel for solidification, in accordance with an embodiment of the present disclosure.

FIG. 17 illustrates the gelation process involving saturated potassium dichromate solution upon addition of sulphuric acid: (a) 1 mL 28% sodium silicate solution (aq.) containing 0.3 g NaOH, (b) 1 mL saturated potassium dichromate solution, (c) 1 mL 50% aqueous NaOH+1 mL saturated potassium dichromate solution and (d) after addition of sulphuric acid for gelation, in accordance with an embodiment of the present disclosure.

FIG. 18 illustrates the solidification process involving saturated potassium dichromate solution upon addition of sulphuric acid and silica gel (chromatographic grade, 60-120 mesh): (a) 1 mL 28% sodium silicate solution (aq.) containing 0.3 g NaOH, (b) 1 mL saturated potassium dichromate solution, (c) 1 mL 50% aqueous NaOH+1 mL saturated potassium dichromate solution and (d) after addition of sulphuric acid for gelation followed by silica gel for solidification, in accordance with an embodiment of the present disclosure.

FIG. 19 illustrates the solidification process involving saturated potassium dichromate solution upon addition of acetic acid and alumina (chromatographic grade, basic, 60-325 mesh): (a) 1 mL 28% sodium silicate solution (aq.) containing 0.3 g NaOH, (b) 1 mL saturated potassium dichromate solution, (c) 1 mL 50% aqueous NaOH+1 mL saturated potassium dichromate solution and (d) after addition of acetic acid for flocculation followed by alumina for solidification, in accordance with an embodiment of the present disclosure.

FIG. 20 illustrates the solidification process involving a mixture of saturated salt (NaCl) and 6% BSA solutions upon addition of acetic acid and alumina (chromatographic grade, basic, 60-325 mesh): (a) 1 mL 28% sodium silicate solution (aq.) containing 0.3 g NaOH, (b) 0.5 mL saturated salt+0.5 mL 6% BSA solutions, (c) 1 mL 50% aqueous NaOH+1 mL saturated salt+6% BSA solutions and (d) after addition of acetic acid for flocculation followed by alumina for solidification, in accordance with an embodiment of the present disclosure.

FIG. 21 illustrates the solidification process involving saturated potassium dichromate solution upon addition of acetic acid and titania (nanopowder, mixture of rutile and anatase): (a) 1 mL 28% sodium silicate solution (aq.) containing 0.3 g NaOH, (b) 1 mL saturated potassium dichromate solution, (c) 1 mL 50% aqueous NaOH+1 mL saturated potassium dichromate solution and (d) after addition of acetic acid for flocculation followed by titania for solidification, in accordance with an embodiment of the present disclosure.

FIG. 22 illustrates the solidification process involving cotton pieces upon addition of acetic acid and silica gel (chromatographic grade, 60-120 mesh): (a) 1 mL 28% sodium silicate solution (aq.) containing 0.3 g NaOH+a piece of cotton, and (b) after addition of acetic acid for flocculation followed by silica gel for solidification, in accordance with an embodiment of the present disclosure.

FIG. 23 illustrates the flocculation process involving tissue paper upon addition of acetic acid: (a) 1 mL 28% sodium silicate solution (aq.) containing 0.3 g NaOH+a piece of tissue paper, and (b) after addition of acetic acid for flocculation, in accordance with an embodiment of the present disclosure.

FIG. 24 illustrates the solidification process involving tissue paper upon addition of acetic acid and silica gel (chromatographic grade, 60-120 mesh): (a) 1 mL 28% sodium silicate solution (aq.) containing 0.3 g NaOH+a piece of tissue paper, and (b) after addition of acetic acid for flocculation followed by silica gel for solidification, in accordance with an embodiment of the present disclosure.

FIG. 25 illustrates the solidification process involving tissue paper upon addition of sulphuric acid and silica gel (chromatographic grade, 60-120 mesh): (a) 1 mL 28% sodium silicate solution (aq.) containing 0.3 g NaOH+a piece of tissue paper, and (b) after addition of sulphuric acid for flocculation followed by silica gel for solidification, in accordance with an embodiment of the present disclosure.

FIG. 26 illustrates the solidification process involving tissue paper upon addition of hydrochloric acid and silica gel (chromatographic grade, 60-120 mesh): (a) 1 mL 28% sodium silicate solution (aq.) containing 0.3 g NaOH+a piece of tissue paper, and (b) after addition of hydrochloric acid for flocculation followed by silica gel for solidification.

FIG. 27 illustrates the solidification process involving needles upon addition of acetic acid and silica gel (chromatographic grade, 60-120 mesh): (a) 1 mL 28% sodium silicate solution (aq.) containing 0.3 g NaOH+a needle, and after addition of acetic acid for flocculation followed by silica gel for solidification, in accordance with an embodiment of the present disclosure.

FIG. 28 illustrates the solidification process involving solid swabs upon addition of acetic acid and silica gel (chromatographic grade, 60-120 mesh): (a) 1 mL 28% sodium silicate solution (aq.) containing 0.3 g NaOH+a swab, and (b) after addition of acetic acid for flocculation followed by silica gel for solidification, in accordance with an embodiment of the present disclosure.

FIG. 29 illustrates the photographs of the petridishes cultured with the samples taken (A,D) as control, (B,E) after addition of aqueous sodium silicate containing NaOH and (C,F) after solidification of an aqueous solution and bacterial broths containing (A-C) E. coli, and (D-F) S. aureus confirming complete disinfection in quantitative experiments, in accordance with an embodiment of the present disclosure.

FIG. 30 illustrates the large-scale solidification process involving a mixture of solid and liquid wastes upon addition of acetic acid and silica gel (chromatographic grade, 60-120 mesh): (a) a mixture of solid and liquid wastes in 1 mL 28% sodium silicate solution (aq.) containing 0.3 g NaOH and (b) after addition of acetic acid for flocculation followed by silica gel (60-120 mesh) for solidification, in accordance with an embodiment of the present disclosure.

FIG. 31 illustrates a prototype of an all-in-one sample collection-disinfection-solidification-disposal device for liquid samples, (a) consisting of four collection vials mounted one on top of the other such that (b) the top vial contains solid material D (silica is shown as an example), the second one with solution C (acetic acid), the third one with collected sample and the bottom one prefilled with the requisite amount of solution A (sodium silicate) containing B (NaOH). Once collected sample is tested, the remaining sample could be initially disinfected by allowing (c) the sample to mix with solution A+B by breaking the junction between third and bottom compartments and (d) flocculation via the addition of solution C by breaking the junction between the second and third compartments (e) followed by solidification via the addition of A by breaking the junction between the top and second compartments, in accordance with an embodiment of the present disclosure.

FIG. 32 illustrates a prototype of an all-in-one sample collection-disinfection-solidification-disposal device for solid samples, (a) consisting of three collection vials mounted one on top of the other such that (b) the top vial contains solid material D (silica is shown as an example), the middle one filled with solution C (acetic acid) and the bottom one prefilled with the requisite amount of solution A containing B. The waste sample could be initially disinfected by (c) mixing the sample with solution A+B followed by (d) flocculation via the addition of solution C by breaking the junction between the middle and bottom compartments and (e) solidification via the addition of material A by breaking the junction between the top and middle compartments.

FIG. 33 (FIG. 33) illustrates the design of the prototype of an all-in-one sample collection-disinfection-solidification-disposal device for liquid samples as shown in FIG. 31, in accordance with an embodiment of the present disclosure.

FIG. 34 illustrates the design of the prototype of an all-in-one sample collection-disinfection-solidification-disposal device for solid samples as shown in FIG. 32, in accordance with an embodiment of the present disclosure.

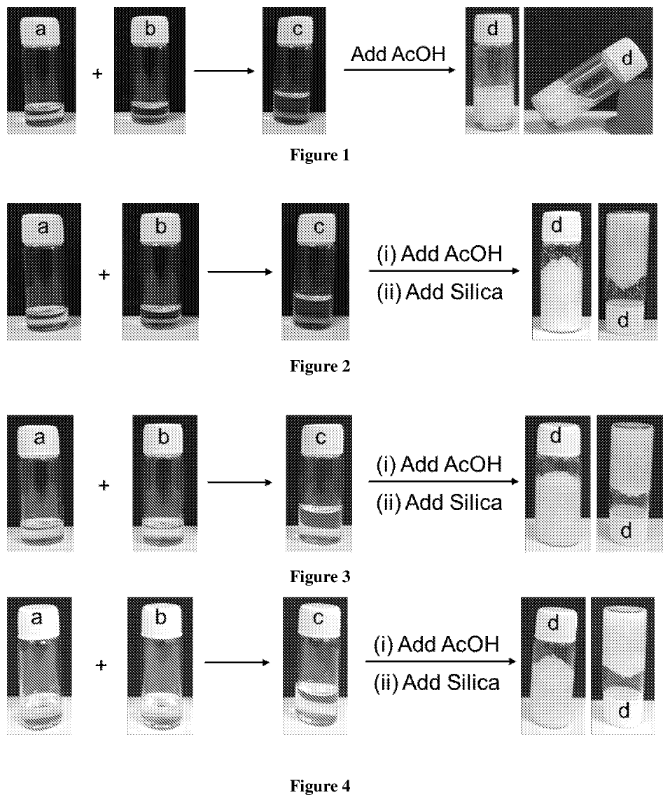

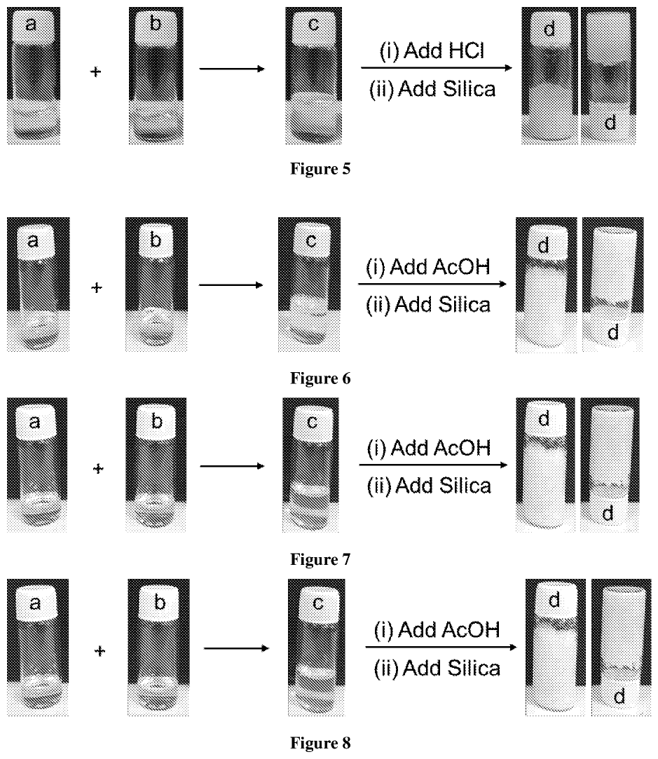

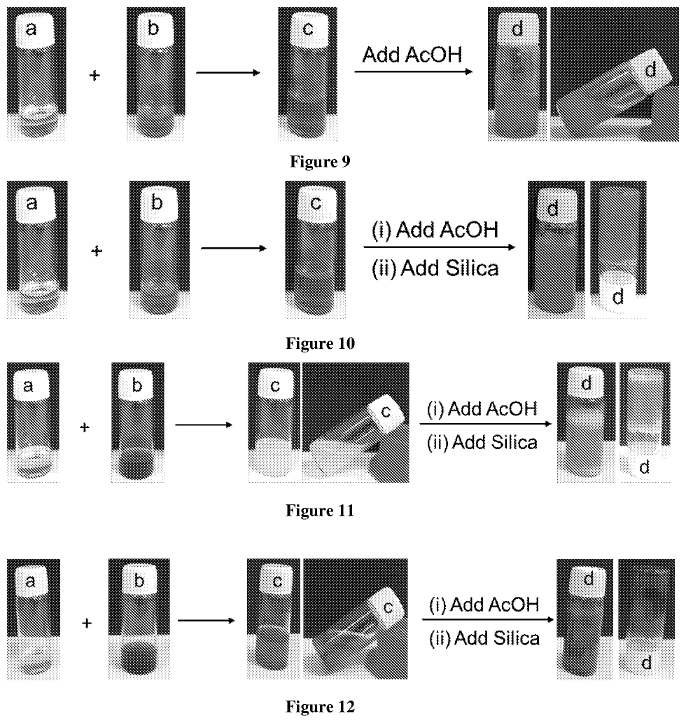

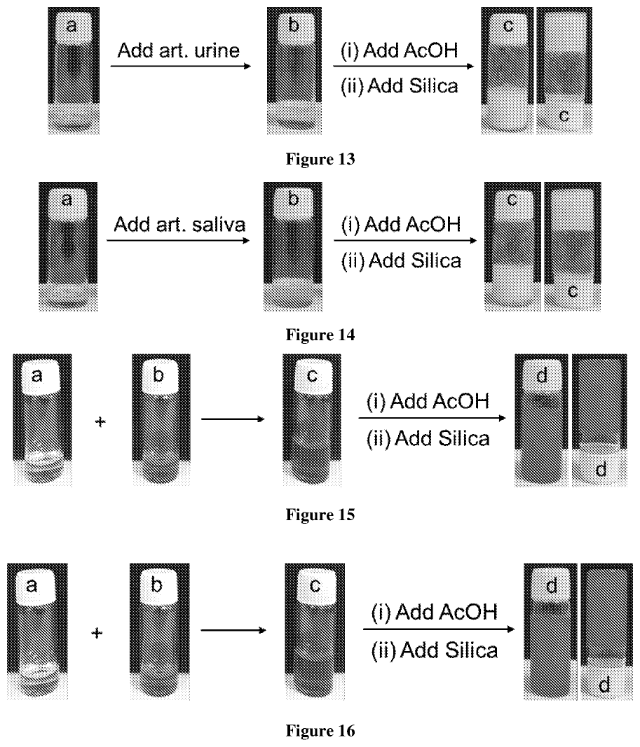

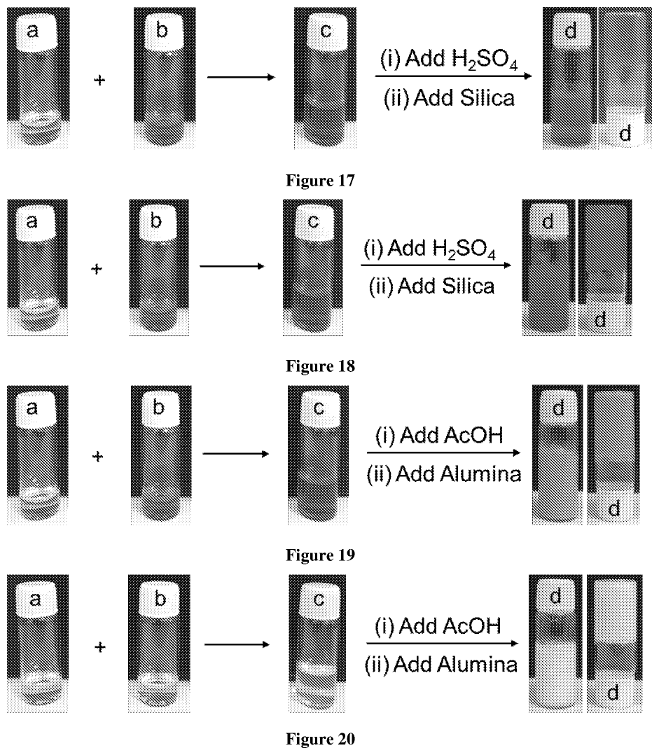

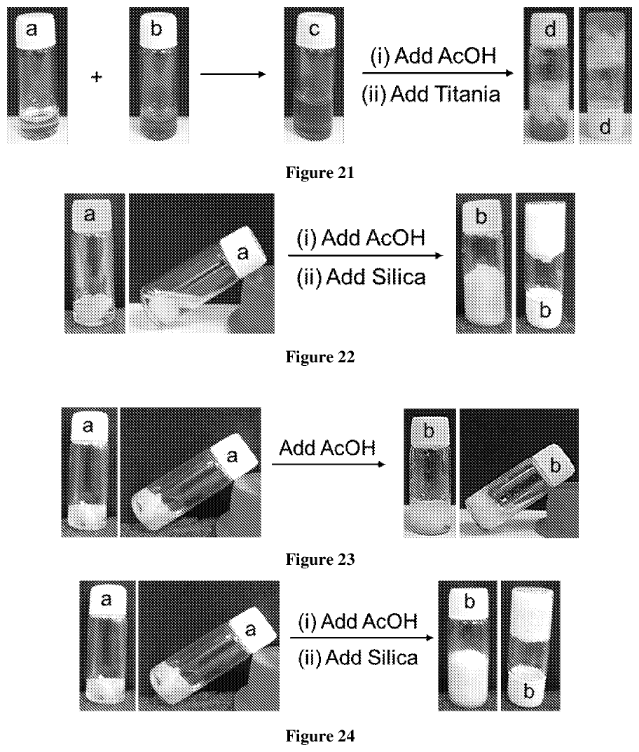

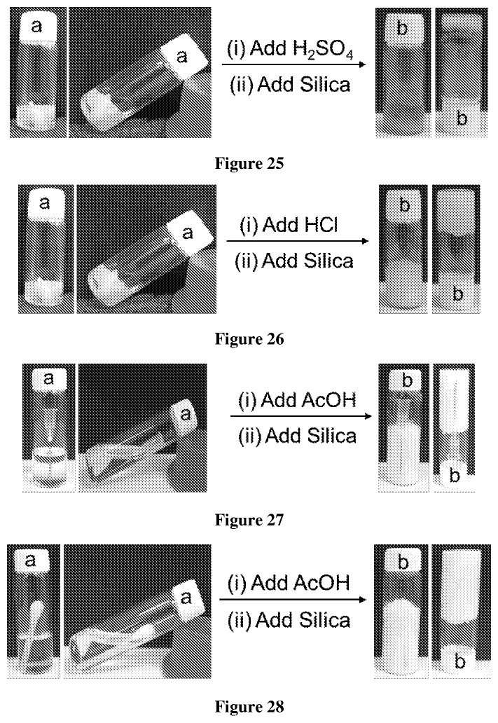

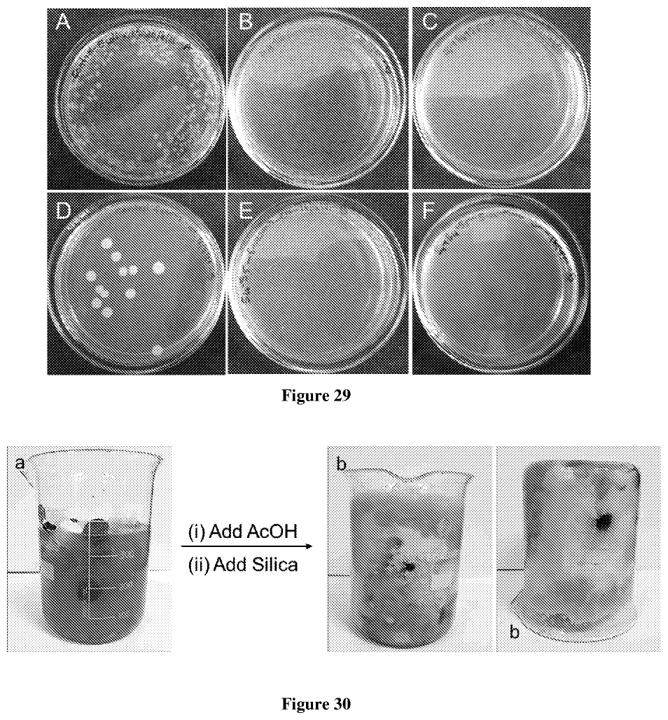

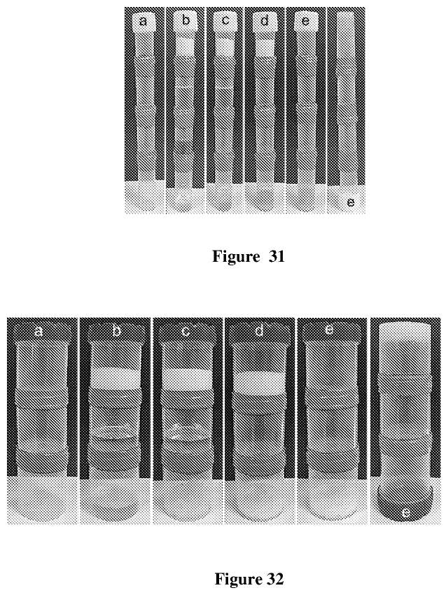

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”. Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight percentage in the range of 0.5-80% should be interpreted to include not only the explicitly recited limits of 0.5-80% but also to include sub-ranges, such as 0.6-70%, 0.5-79%, 1-60%, and so forth, as well as individual amounts, within the specified ranges, such as 20%, 40%, 55.2%, 60%, 80% and so on.

This section describes the present invention in preferred embodiments in detail. The attached illustrations/drawings are intended for the purpose of describing and understanding the preferred embodiments in detail and not to limit the invention or its scope or both thereto.

Previously, it has been found that adding a poly-amino acid as its aqueous solution to a stable nanomaterial sol in water leads to instantaneous flocculation and the said flocculation process could further be controlled to effect gelation or solidification under carefully controlled conditions. However, the use of sols and polyamino acids are not very effective for long term treatment and resting of biomedical waste. Also, there is a need for stopping the process in a flocculated state or continue to the solidification state depending on the type of biomedical waste. The present invention provides a process for the disinfection and flocculation or solidification of biomedical waste, that involves the use of alkaline solution of metal silicates, organic or inorganic acids as flocculating agent and solid powders of a solidifying agent, which when subjected to mixing with solid or fluid waste samples at a defined volumetric and/or weighted composition leads to instantaneous flocculation or solidification with up to 100% microbial disinfection.

The present invention provides a disinfection-flocculation-solidification process for the preparation for disposal of solid and fluid wastes collected in a collection vessel at point of care, combined with the destruction, disinfection or deactivation of infectious agents including microorganisms inter alia bacteria, fungus etc., viruses and other toxins, whereby the disposal including treatment, handling and transportation are deemed easier, safer and cost-effective. Solidification reduces the risk of spills and aerosolization, whereas complete pathogenic disinfection allows to dispose of the wastes thereof as non-regulated medical waste, which is less expensive than red-bagging. Segregation, transportation and incineration of such disinfected medical wastes are easier, safer and decrease medical waste disposal costs for a healthcare facility.

Another object of the present invention comprises the addition of an organic or inorganic acid to an alkaline solution of metal silicates containing the biomedical entity to be disinfected leading to flocculation and further addition of oxides or phosphates of transition metals inter alia titanium, aluminium, silicon, zinc, cerium or lanthanum, with or without a binder, wherein the silicate aqueous solution is basified to an alkaline pH using a base, such that the concentration of the base is 0.1-90% w/v in water, more preferably 0.1-1 g/mL in water, the acid is any organic or inorganic acid chosen from acetic, hydrochloric, sulphuric or phosphoric acids and the solid oxide or phosphate powders are added at a minimum of 0.5% (w/v) and a maximum of 1000% (w/v) of the total aqueous volume, resulting in instantaneous disinfection followed by instantaneous flocculation/solidification.

The present invention intends to offer a self-disinfecting solidification process for the treatment and disposal of biomedical waste. The treatment process disclosed herein involves an aqueous solution of metal silicates such as sodium or potassium silicate at a concentration of 0.5-80%, preferably 20-40% in water, the basifying agent is selected from hydroxides of alkali or alkaline earth metals, basic salts of metals and organic cations, preferably sodium or potassium hydroxide, leading to a final pH in the range 9-14 when added to solution the silicates in the range 0.1-5 g/mL of the silicate, an acid with a general formula HₙX, wherein X is selected from a group of anions inter alia halides, acetate, sulphate, phosphate, etc. and n is an integer such that 1≤n≤3, the solidifying agent, with or without a binder, is selected from silica, preferably chromatography grade silica gel powder of 60-400 mesh size, alumina, preferably chromatography grade alumina powder of 60-200 mesh size, titania, preferably pigment grade titania in its rutile or anatase forms or a mixture of rutile and anatase forms, zinc oxide, preferably industrial grade zinc oxide in its powder form having particle size <500 μm, lanthanum or cerium phosphate as nanopowders, added at defined volumetric and/or weighted composition leading to instantaneous solidification with up to 100% microbial disinfection.

In specific embodiments, the invention relates to providing a flocculated or non-pourable environment for fluid medical wastes inter alia salt, sugar, saliva, urine, blood, hapital chemicals, etc. wherein risks related to spillage and occupational exposure are minimized, and further to the treatment of solid medical wastes inter alia cotton, tissue paper, swabs, needles, etc., wherein the risks related to accumulation of untreated and infected samples are minimized or a mixture of solid and liquid wastes added with >99.9% microbial disinfection.

Another aspect of the present invention intends to disclose the volumetric composition of an aqueous solution of a metal silicate containing a pH regulating base or alkali for complete disinfection of fluid or solid medical waste followed by the addition of an acid for flocculation or an oxide/phosphate based solid powder, as a single or plurality of the said powders, for instantaneous solidification of solid or fluid samples containing proteins, microbial cultures, salt or metal ions in high concentrations.

Another aspect of the present invention is directed to creating all-in-one sample collection-disinfection-solidification devices of requisite dimensions capable of collecting the solid or liquid sample, flocculating/gelating/solidifying the samples as and when required and disinfecting the same for preparation for its disposal. and immobilizing them as and when required with prior pathogenic disinfection for preparation for its disposal.

In an embodiment the present invention provides a process for disinfection followed by in situ flocculation and solidification by disinfection-flocculation-solidification and disposal system, wherein disinfection composition comprising of a four chemical components A, B, C and D, wherein: a) A is a silicate salt of an alkali metal selected from the group consisting of sodium, potassium, and combinations thereof in its 20-40% aqueous solution at a concentration of 0.5-80% (w/v); b) B is a base at a concentration of 0.1-90% w/v added to an aqueous solution of A; c) C is an organic or inorganic acid miscible completely in water; and d) D is a solidifying agent selected from an oxide or a phosphate based powder inter alia oxides/phosphates of silicon, titanium, zinc, aluminum or lanthanide elements such as cerium or lanthanum.

In an embodiment the present invention provides a process for disinfection followed by in situ flocculation and solidification by disinfection-flocculation-solidification and disposal system as disclosed herein, wherein B, the base is selected from group consisting of hydroxides of alkali or alkaline earth metals selected from the group consisting of sodium or potassium hydroxide, basic salts of metals and organic cations, leading to a final pH in the range 9-14 when added to solution A, in the range 0.1-5 g/mL of A.

In further embodiment the present invention provides a process for disinfection followed by in situ flocculation and solidification by disinfection-flocculation-solidification and disposal system as disclosed herein, wherein C is an organic or inorganic acid with a general formula HₙX, wherein X is selected from a group of anions inter alia halides, acetate, sulphate, and phosphate, and n is an integer such that 1≤n≤3.

In another embodiment the present invention provides a process for disinfection followed by in situ flocculation and solidification by disinfection-flocculation-solidification and disposal system as disclosed herein, wherein the solidifying agent, with or without a binder, is selected from chromatography grade silica gel powder of 60-400 mesh size, chromatography grade alumina powder of 60-200 mesh size, pigment grade titania in its rutile or anatase forms or a mixture of rutile and anatase forms, industrial grade zinc oxide in its powder form having particle size <500 μm, or lanthanum or cerium phosphate as nanopowders.

In an embodiment the present invention provides a process for disinfection followed by in situ flocculation and solidification by disinfection-flocculation-solidification and disposal system, comprising the steps of: a) addition of B to an aqueous solution of A; b) addition of a biomedical waste to be disinfected to the said aqueous solution as prepared in step (a); c) homogeneous mixing of the mixture as in (b) and/or resting for 10-30 min, wherein the obtained mixture is characterized as flocculated; and d) addition of material C and material D as its solid powder followed by mixing and/or resting, wherein the obtained mixture is characterized as solidified.

In an embodiment the present invention provides a process for disinfection followed by in situ flocculation and solidification by disinfection-flocculation-solidification and disposal system as disclosed herein, wherein amount of the waste added is less than 1:1000 (v/v) of solution B for liquid waste and any immersible amount of solid waste or a mixture thereof.

In an embodiment the present invention provides a process for disinfection followed by in situ flocculation and solidification by disinfection-flocculation-solidification and disposal system as disclosed herein, wherein C is glacial acetic acid and the amount of C added is 0.1-3 mL per mL of the total aqueous mixture obtained in step (b).

In an embodiment the present invention provides a process for disinfection followed by in situ flocculation and solidification by disinfection-flocculation-solidification and disposal system as disclosed herein, wherein solid A is added at a minimum of 1% (w/v) and a maximum of 500% (w/v) of the total aqueous volume in the mixture obtained in step (c).

In an embodiment the present invention provides a process for disinfection followed by in situ flocculation and solidification by disinfection-flocculation-solidification and disposal system as disclosed herein, wherein the biomedical waste samples used in step (b) is selected from the group consisting of salt, sugar, metal salts and complexes, aqueous waste, hospital chemicals such as iodine, saliva, urine, blood or any solid sample, inter alia cotton, tissue paper, needle, syringes or swabs alone or in combination thereof, whereby disinfection is effected by the high pH of solution A containing B.

In an embodiment the present invention provides a process for disinfection followed by in situ flocculation and solidification by disinfection-flocculation-solidification and disposal system as disclosed herein, wherein said process assists in either flocculation leading to soft flocculated solids when terminated at step (c) or solidification leading to hard solids when continued to step (d).

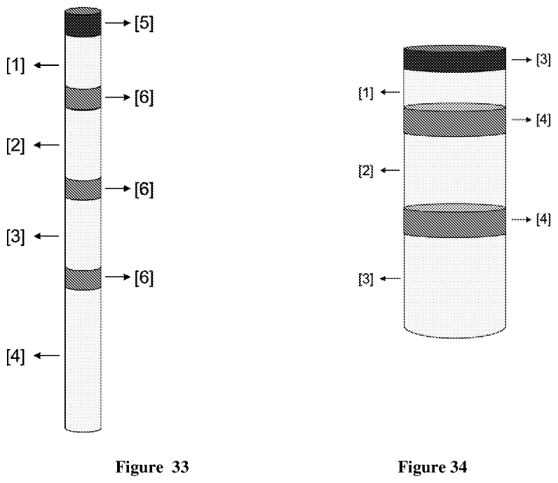

In an embodiment the present invention provides disinfection-flocculation-solidification and disposal device filled with the disinfection composition comprising four chemical components A, B, C and D as disclosed herein, the device comprising of: a) an upper container or compartment system [1, FIG. 33]; b) a second container or compartment system [2, FIG. 33]; c) a third container or compartment system [3, FIG. 33]; d) a bottom container or compartment system [4, FIG. 33]; e) a screw cap [5, FIG. 33] connected to the upper container or compartment system; and f) three breakable screw-caps [6, FIG. 33], one connected between the upper and the second container or compartment systems, another connected between the second and the third container or compartment systems and a third connected to the third and the bottom container or compartment systems.

In an embodiment the present invention provides disinfection-flocculation-solidification and disposal device as disclosed herein, wherein the upper container or compartment system is filled with solid powder of material D.

In an embodiment the present invention provides disinfection-flocculation-solidification and disposal device as disclosed herein, wherein the second container or compartment system is filled with solution C.

In an embodiment the present invention provides disinfection-flocculation-solidification and disposal device as disclosed herein, wherein the third container or compartment system is filled with the biomedical waste sample.

In an embodiment the present invention provides disinfection-flocculation-solidification and disposal device as disclosed herein, wherein the bottom container or compartment system is filled with the aqueous solution of A mixed with B as disclosed herein.

In an embodiment the present invention provides disinfection-flocculation-solidification and disposal device as disclosed herein, wherein the biomedical sample is solid or liquid waste or their mixture.

Although the subject matter has been described with reference to specific embodiments, this description is not meant to be construed in a limiting sense. Various modifications of the disclosed embodiments, as well as alternate embodiments of the subject matter, will become apparent to persons skilled in the art upon reference to the description of the subject matter. It is therefore contemplated that such modifications can be made without departing from the spirit or scope of the present subject matter as defined.

### EXAMPLES

The disclosure will now be illustrated with the working examples, which is intended to illustrate the working of disclosure and not intended to take restrictively to imply any limitations on the scope of the present disclosure. Unless defined otherwise, all technical and scientific terms used herein have the same meaning as commonly understood to one ordinary person skilled in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

Following examples are given by way of illustration and therefore should not be construed to limit the scope of the invention.

### Example 1. Flocculation of Aqueous Waste Using Sodium Silicate and Acid

To an aqueous solution of sodium silicate (20-40%), sodium hydroxide (300 mg/mL) was added. Aqueous waste (1:1) was added to the above solution and mixed well. Acetic acid was added dropwise and instantaneous flocculation was observed. Sulfuric acid, hydrochloric acid or phosphoric acid was also used instead of acetic acid.

### Example 2. Solidification of Aqueous Waste Using Sodium Silicate, Acid and Silica Gel Powder (60-120, 100-200 or 230-400 Mesh)

To an aqueous solution of sodium silicate (20-40%), sodium hydroxide (300 mg/mL) was added. Aqueous waste (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 3. Solidification of Concentrated Salt Solution Using Sodium Silicate, Acid and Silica Gel Powder (60-120, 100-200 or 230-400 Mesh)

To an aqueous solution of sodium silicate (20-40%), sodium hydroxide (300 mg/mL) was added. A saturated aqueous solution of sodium chloride (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 4. Solidification of Concentrated Sugar Solution Using Sodium Silicate, Acid and Silica Gel Powder (60-120, 100-200 or 230-400 Mesh)

To an aqueous solution of sodium silicate (20-40%), sodium hydroxide (300 mg/mL) was added. A saturated aqueous solution of sucrose (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 5. Solidification of a Mixture of Concentrated Salt and Sugar Solutions Using Sodium Silicate, Acid and Silica Gel Powder (60-120, 100-200 or 230-400 Mesh)

To an aqueous solution of sodium silicate (20-40%), sodium hydroxide (300 mg/mL) was added. A mixture of saturated aqueous solutions of sodium chloride and sucrose (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 6. Solidification of Aqueous Waste Containing Proteins Using Sodium Silicate, Acid and Silica Gel Powder (60-120, 100-200 or 230-400 Mesh)

To an aqueous solution of sodium silicate (20-40%), sodium hydroxide (300 mg/mL) was added. A 6% aqueous solution of BSA (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 7. Solidification of Concentrated Salt Solution Containing Proteins Using Sodium Silicate, Acid and Silica Gel Powder (60-120, 100-200 or 230-400 Mesh)

To an aqueous solution of sodium silicate (20-40%), sodium hydroxide (300 mg/mL) was added. A saturated aqueous solution of sodium chloride containing 6% BSA (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 8. Flocculation of Aqueous Solution Containing Metal Ions and Harsh Oxidising Agent Using Sodium Silicate and Acid

To an aqueous solution of sodium silicate (20-40%), sodium hydroxide (300 mg/mL) was added. A saturated aqueous solution of potassium dichromate (1:1) was added to the above solution and mixed well. Acetic acid was added dropwise and instantaneous flocculation was observed. Sulfuric acid, hydrochloric acid or phosphoric acid was also used instead of acetic acid.

### Example 9. Solidification of Aqueous Solution Containing Metal Ions and Harsh Oxidising Agent Using Sodium Silicate, Acid and Silica Gel Powder (60-120, 100-200 or 230-400 Mesh)

To an aqueous solution of sodium silicate (20-40%), sodium hydroxide (300 mg/mL) was added. A saturated aqueous solution of potassium dichromate (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 10. Solidification of Aqueous Waste Containing Hospital Chemicals Using Sodium Silicate, Acid and Silica Gel Powder (60-120, 100-200 or 230-400 Mesh)

To an aqueous solution of sodium silicate (20-40%), sodium hydroxide (300 mg/mL) was added. An aqueous solution of iodine (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 11. Solidification of Aqueous Wastes Using Sodium Silicate, Acid and Alumina Powder (60-400 Mesh)

To an aqueous solution of sodium silicate (20-40%), sodium hydroxide (300 mg/mL) was added. Aqueous waste (1:1) was added to the above solution and mixed well. Acetic acid was added dropwise and instantaneous flocculation was observed. Addition of alumina powder resulted in instantaneous solidification. Sulfuric acid, hydrochloric acid or phosphoric acid was also used instead of acetic acid.

### Example 12. Solidification of Aqueous Wastes Using Sodium Silicate, Acid and Titania Powder (Mixture of Anatase and Rutile)

To an aqueous solution of sodium silicate (20-40%), sodium hydroxide (300 mg/mL) was added. Aqueous waste (1:1) was added to the above solution and mixed well. Acetic acid was added dropwise and instantaneous flocculation was observed. Addition of titania powder resulted in instantaneous solidification. Sulfuric acid, hydrochloric acid or phosphoric acid was also used instead of acetic acid.

### Example 13. Solidification of Aqueous Wastes Using Sodium Silicate, Acid and Zinc Oxide Powder (Particle Size <500 μm)

To an aqueous solution of sodium silicate (20-40%), sodium hydroxide (300 mg/mL) was added. Aqueous waste (1:1) was added to the above solution and mixed well. Acetic acid was added dropwise and instantaneous flocculation was observed. Addition of zinc oxide powder resulted in instantaneous solidification. Sulfuric acid, hydrochloric acid or phosphoric acid was also used instead of acetic acid.

### Example 14. Solidification of Aqueous Wastes Using Sodium Silicate, Acid and Metal Phosphate Powder

To an aqueous solution of sodium silicate (20-40%), sodium hydroxide (300 mg/mL) was added. Aqueous waste (1:1) was added to the above solution and mixed well. Acetic acid was added dropwise and instantaneous flocculation was observed. Addition of lanthanum or cerium phosphate powder resulted in instantaneous solidification. Sulfuric acid, hydrochloric acid or phosphoric acid was also used instead of acetic acid.

### Example 15. Preparation of Artificial Saliva

Artificial saliva was prepared according to the following two procedures: (i) Mixing 1.5 mM Ca(NO₃)₂, 0.90 mM KH₂PO₄, 130 mM KCl and 60 mM Tris buffer at pH 7.4 (Reference may be made to: Kirkham, J.; et al., Self-assembling peptide scaffolds promote enamel remineralization, J. Dental Res. 2007, 86, 426-430). (ii) Mixing sodium chloride (0.06 g), potassium chloride (0.072 g), calcium chloride dihydrate (0.022 g), potassium dihydrogen phosphate (0.068 g), disodium hydrogen phosphate dodecahydrate (0.086 g), potassium thiocyanate (0.006 g), sodium hydrogen carbonate (0.15 g), and citric acid (0.003 g) in 100 mL distilled water at pH 6.5 (Reference may be made to: Duffó, G. S.; et al., Development of an artificial saliva solution for studying the corrosion behavior of dental alloys. Corrosion 2004, 60, 594-602).

### Example 16. Solidification of Artificial Saliva Using Sodium Silicate, Acid Silica Gel Powder (60-120, 100-200 or 230-400 Mesh)

To an aqueous solution of sodium silicate (20-40%), sodium hydroxide (300 mg/mL) was added. Artificial saliva (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 17. Solidification of Artificial Saliva Using Sodium Silicate, Acid and Alumina Powder (60-400 Mesh)

To an aqueous solution of sodium silicate (20-40%), sodium hydroxide (300 mg/mL) was added. Artificial saliva (1:1) was added to the above solution and mixed well. Acetic acid was added dropwise and instantaneous flocculation was observed. Addition of alumina powder resulted in instantaneous solidification. Sulfuric acid, hydrochloric acid or phosphoric acid was also used instead of acetic acid.

### Example 18. Solidification of Artificial Saliva Using Sodium Silicate, Acid and Titania Powder (Mixture of Anatase and Rutile)

To an aqueous solution of sodium silicate (20-40%), sodium hydroxide (300 mg/mL) was added. Artificial saliva (1:1) was added to the above solution and mixed well. Acetic acid was added dropwise and instantaneous flocculation was observed. Addition of titania powder resulted in instantaneous solidification. Sulfuric acid, hydrochloric acid or phosphoric acid was also used instead of acetic acid.

### Example 19. Solidification of Artificial Saliva Using Sodium Silicate, Acid and Zinc Oxide Powder (Particle Size <500 μm)

To an aqueous solution of sodium silicate (20-40%), sodium hydroxide (300 mg/mL) was added. Artificial saliva (1:1) was added to the above solution and mixed well. Acetic acid was added dropwise and instantaneous flocculation was observed. Addition of zinc oxide powder resulted in instantaneous solidification. Sulfuric acid, hydrochloric acid or phosphoric acid was also used instead of acetic acid.

### Example 20. Preparation of Artificial Urine

To 75 mL of distilled water in a container, urea (1.82 g) was added and shaken well to dissolve. Sodium chloride (0.75 g), potassium chloride (0.45 g) and sodium phosphate (0.48 g) were further added to the above mixture and mixed well until dissolved. The pH was adjusted to be between 5 and 7. Creatinine (200 mg) and albumin powder (5 mg) were added and mixed gently. The artificial urine thus obtained was further spiked with a few mg of glucose before each experiment.

### Example 21. Solidification of Artificial Urine Using Sodium Silicate, Acid and Silica Gel Powder (60-120, 100-200 or 230-400 Mesh)

To an aqueous solution of sodium silicate (20-40%), sodium hydroxide (300 mg/mL) was added. Artificial urine (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 22. Solidification of Artificial Urine Using Sodium Silicate, Acid and Alumina Powder (60-400 Mesh)

To an aqueous solution of sodium silicate (20-40%), sodium hydroxide (300 mg/mL) was added. Artificial urine (1:1) was added to the above solution and mixed well. Acetic acid was added dropwise and instantaneous flocculation was observed. Addition of alumina powder resulted in instantaneous solidification. Sulfuric acid, hydrochloric acid or phosphoric acid was also used instead of acetic acid.

### Example 23. Solidification of Artificial Urine Using Sodium Silicate, Acid and Titania Powder (Mixture of Anatase and Rutile)

To an aqueous solution of sodium silicate (20-40%), sodium hydroxide (300 mg/mL) was added. Artificial urine (1:1) was added to the above solution and mixed well. Acetic acid was added dropwise and instantaneous flocculation was observed. Addition of titania powder resulted in instantaneous solidification. Sulfuric acid, hydrochloric acid or phosphoric acid was also used instead of acetic acid.

### Example 24. Preparation of Artificial Blood

A 6% solution of BSA was prepared in distilled water. A small amount of an iron(II) complex was added to mimic heme and impart color.

### Example 25. Solidification of Artificial Blood Using Sodium Silicate, Acid and Silica Gel Powder (60-120, 100-200 or 230-400 Mesh)

To an aqueous solution of sodium silicate (20-40%), sodium hydroxide (300 mg/mL) was added. Artificial blood (1:1) was added to the above solution and mixed well. Acetic acid was added dropwise and instantaneous flocculation was observed. Addition of silica gel powder resulted in instantaneous solidification. Sulfuric acid, hydrochloric acid or phosphoric acid was also used instead of acetic acid.

### Example 26. Solidification of Artificial Blood Using Sodium Silicate, Acid and Alumina Powder (60-400 Mesh)

To an aqueous solution of sodium silicate (20-40%), sodium hydroxide (300 mg/mL) was added. Artificial blood (1:1) was added to the above solution and mixed well. Acetic acid was added dropwise and instantaneous flocculation was observed. Addition of alumina powder resulted in instantaneous solidification. Sulfuric acid, hydrochloric acid or phosphoric acid was also used instead of acetic acid.

### Example 27. Solidification of Artificial Blood Using Sodium Silicate, Acid and Titania Powder (Mixture of Anatase and Rutile)

To an aqueous solution of sodium silicate (20-40%), sodium hydroxide (300 mg/mL) was added. Artificial blood (1:1) was added to the above solution and mixed well. Acetic acid was added dropwise and instantaneous flocculation was observed. Addition of titania powder resulted in instantaneous solidification. Sulfuric acid, hydrochloric acid or phosphoric acid was also used instead of acetic acid.

### Example 28. Immobilization of a Solid Swab Using Sodium Silicate, Acid and Silica Gel (60-400 Mesh), Alumina (60-400 Mesh), Titania (Mixture of Anatase and Rutile) or Zinc Oxide (Particle Size <500 μm) Powders

In an aqueous solution of sodium silicate containing sodium hydroxide (300 mg/mL) in an 8 mL glass vial, a piece of swab (4 cm) was immersed. It was mixed well, and acetic acid was added dropwise, resulting in flocculation. Solid powder of silica gel (60-400 mesh), alumina (60-400 mesh), titania (mixture of anatase and rutile) or zinc oxide (particle size <500 m) was then added, resulting in instantaneous solidification. Sulfuric acid, hydrochloric acid or phosphoric acid was also used instead of acetic acid.

### Example 29. Immobilization of a Syringe Needle Using Sodium Silicate, Acid and Silica Gel (60-400 Mesh), Alumina (60-400 Mesh), Titania (Mixture of Anatase and Rutile) or Zinc Oxide (Particle Size <500 μm) Powders

In an aqueous solution of sodium silicate containing sodium hydroxide (300 mg/mL) in an 8 mL glass vial, a needle (4-6 cm) was immersed. It was mixed well, and acetic acid was added dropwise, resulting in flocculation. Solid powder of silica gel (60-400 mesh), alumina (60-400 mesh), titania (mixture of anatase and rutile) or zinc oxide (particle size <500 m) was then added, resulting in instantaneous solidification. Sulfuric acid, hydrochloric acid or phosphoric acid was also used instead of acetic acid.

### Example 30. Immobilization of Cotton Waste Using Sodium Silicate, Acid and Silica Gel (60-400 Mesh), Alumina (60-400 Mesh), Titania (Mixture of Anatase and Rutile) or Zinc Oxide (Particle Size <500 μm) Powders

To an aqueous solution of sodium silicate containing sodium hydroxide (300 mg/mL) in an 8 mL glass vial, a piece of cotton was added. It was mixed well, and acetic acid was added dropwise, resulting in flocculation. Solid powder of silica gel (60-400 mesh), alumina (60-400 mesh), titania (mixture of anatase and rutile) or zinc oxide (particle size <500 m) was then added, resulting in instantaneous solidification. Sulfuric acid, hydrochloric acid or phosphoric acid was also used instead of acetic acid

### Example 31. Immobilization of Tissue Paper Using Sodium Silicate, Acid and Silica Gel (60-400 Mesh), Alumina (60-400 Mesh), Titania (Mixture of Anatase and Rutile) or Zinc Oxide (Particle Size <500 μm) Powders

To an aqueous solution of sodium silicate containing sodium hydroxide (300 mg/mL) in an 8 mL glass vial, a piece of tissue paper was added. It was mixed well, and acetic acid was added dropwise, resulting in flocculation. Solid powder of silica gel (60-400 mesh), alumina (60-400 mesh), titania (mixture of anatase and rutile) or zinc oxide (particle size <500 m) was then added, resulting in instantaneous solidification. Sulfuric acid, hydrochloric acid or phosphoric acid was also used instead of acetic acid.

### Example 32. Immobilization of Large Scale Mixed Waste Using Sodium Silicate, Acid and Silica Gel (60-400 Mesh), Alumina (60-400 Mesh), Titania (Mixture of Anatase and Rutile) or Zinc Oxide (Particle Size <500 μm) Powders

To an aqueous solution of sodium silicate containing sodium hydroxide (300 mg/mL) in a glass beaker, a mixture of different wastes (solid and liquid—syringe, needle, swab, cotton, tissue, artificial urine, blood and saliva, iodine, potassium dichromate, salt, sugar, etc.) was added. It was mixed well acetic acid was added dropwise, resulting in flocculation. Solid powder of silica gel (60-400 mesh), alumina (60-400 mesh), titania (mixture of anatase and rutile) or zinc oxide (particle size <500 m) was then added, resulting in instantaneous solidification. Sulfuric acid, hydrochloric acid or phosphoric acid was also used instead of acetic acid.

### Example 33. Antimicrobial Studies

Cultures of Escherichia coli and Staphylococcus aureus were prepared in Luria Bertiani (LB) medium and taken for test at 18 h. old stage where the colony forming units (cfus) are approximately 1-3×10⁶ per millilitre for E. coli or S. aureus. (previously standardized based on optical densities at 600 nm). 1 mL of an aqueous solution of sodium silicate containing sodium hydroxide (300 mg/mL) was added to 1 mL of the bacterial broth (spiking solution) and mixed by swirling the bottle. Samples were taken for analysis after regular intervals of time. Acetic acid was then added dropwise followed by solid powder of silica gel (60-120 mesh) was added to effect instantaneous solidification. Samples were further taken for analysis after regular intervals of time. All samples were taken as diluted 10× in sterile saline and 100 μL of the diluted solution was plated onto LB agar plates and incubated over night at 37° C. Parallelly, the original bacterial suspension was diluted serially in sterile saline and 100 μL of the appropriate dilutions were plated on LB agar plates and incubated as for the test sample that served as controls. Colonies were counted the next day and based on applied dilution, the number of CFUs/mL of the original bacterial suspension added to the sol and the CFUs in the gelled disinfectant were calculated. Efficiency was calculated as follows: [(No of CFUs in Bacterial suspension−No of CFUs in the gelled disinfectant)/No of CFUs in Bacterial suspension]×100 and expressed in %.

### Example 34. Prototype for All-In-One Sample Collection-Disinfection-Disposal Devices for Fluid Samples

An all-in-one sample collection-disinfection-disposal device for fluid samples was prototyped as follows: Four plastic collection vials were mounted one on top of the other such that the top vial contained solid powder of silica gel (60-400 mesh), alumina (60-400 mesh), titania (mixture of anatase and rutile), zinc oxide (particle size <500 μm) or phosphates of cerium or lanthanum, the second one contained acetic, sulphuric, hydrochloric or phosphoric acid, the third one for sample collection and the bottom one prefilled with the requisite amount of an aqueous solution of sodium silicate containing sodium hydroxide (300 mg/mL). The design allowed the top compartments to be unscrewed and the samples could be collected in the third compartment. Once collected sample was tested, the remaining sample was disinfected by initially allowing the sample to mix with the alkaline sodium silicate solution in the bottom container by breaking the junction between the third and bottom compartments followed by flocculation using acid in the second compartment by breaking the junction between the second and third compartments. Addition of the corresponding solid powder from the top compartment by breaking the junction between the top and second compartments resulted in solidification. The mixing allows for complete pathogenic disinfection as evidenced in Example 33.

### Example 35. Prototype for All-In-One Sample Collection-Disinfection-Disposal Devices for Solid Samples

An all-in-one sample collection-disinfection-disposal device for solid samples was prototyped as follows: A plastic collection container for solid samples (Eg.: cotton waste) was mounted on its top with two plastic vials such that the top vial contained silica gel (60-400 mesh), alumina (60-400 mesh), titania (mixture of anatase and rutile) or zinc oxide (particle size <500 m), the middle one contained acetic, sulphuric, hydrochloric or phosphoric acid and the bottom one was prefilled with the requisite amount of an aqueous solution of sodium silicate containing sodium hydroxide (300 mg/mL). The design allowed the top compartments to be unscrewed and the solid samples could be collected in the bottom compartment. Once solid samples were collected in the bottom container, it was disinfected and flocculated by allowing the alkaline sample in the bottom compartment to mix with the corresponding acid by breaking the junction between the middle and the bottom compartments. Addition of the solid powder by breaking the junction between the top and middle compartments resulted in solidification. The mixing allows for complete pathogenic disinfection as evidenced in Example 33.

### Advantages of the Invention

The present invention provides a process for disinfection followed by in situ flocculation and solidification by disinfection-flocculation-solidification and disposal system which exhibits inherent antimicrobial activity. The process of the present invention is an instantaneous process for disinfection and solidification upon mixing. The process also provides >99.9% microbial disinfection within 1 minute. The process of present invention provides possibilities to stop at an in situ flocculated state for easy retrieval and recycling. Further the process of present invention reduces risks of spillage and occupational exposure. Also the process of present invention allows to dispose the waste as non-regulated medical waste. The provided process of the present invention is applicable to both fluid as well as solid medical waste decontamination. The process of the present invention is a safer, easier, and cost-effective due to use of precursor materials and is adaptable to manage any amount of fluidic waste. Additionally the present invention provides a process which is uninterrupted and there is no interference from proteins, metal ions, salt, or other impurities.

1. A process for disinfection followed by in-sit in situ flocculation and solidification by disinfection-flocculation-solidification and disposal system, wherein disinfection composition comprising of a four chemical components A, B, C and D, wherein:
(a) A is a silicate salt of an alkali metal selected from the group consisting of sodium, potassium, and combinations thereof in its 20-40% aqueous solution at a concentration of 0.5-80% (w/v);
(b) B is a base at a concentration of 0.1-90% w/v added to an aqueous solution of A;
(c) C is an organic or inorganic acid miscible completely in water; and
(d) D is a solidifying agent selected from an oxide or a phosphate based powder inter alia oxides/phosphates of silicon, titanium, zinc, aluminum, or lanthanide elements such as cerium or lanthanum.

2. The process for disinfection followed by in situ flocculation and solidification as claimed in claim 1, wherein B, the base is selected from group consisting of hydroxides of alkali or alkaline earth metals selected from the group consisting of sodium or potassium hydroxide, basic salts of metals and organic cations, leading to a final pH in the range 9-14 when added to solution A, in the range 0.1-5 g/mL of A.

3. The process for disinfection followed by in situ flocculation and solidification as claimed in claim 1, wherein C is an organic or inorganic acid with a general formula HₙX, wherein X is selected from a group of anions inter alia halides, acetate, sulphate, and phosphate, and n is an integer such that 1≤n≤3.

4. The process for disinfection followed by in situ flocculation and solidification as claimed in claim 1, wherein the solidifying agent, with or without a binder, is selected from chromatography grade silica gel powder of 60-400 mesh size, chromatography grade alumina powder of 60-200 mesh size, pigment grade titania in its rutile or anatase forms or a mixture of rutile and anatase forms, industrial grade zinc oxide in its powder form having particle size <500 μm, or lanthanum or cerium phosphate as nanopowders.

5. The process for disinfection-flocculation-solidification as claimed in claim 1, comprising the steps of:
(a) addition of B to an aqueous solution of A;
(b) addition of a biomedical waste to be disinfected to the said aqueous solution as prepared in step (a);
(c) homogeneous mixing of the mixture as in (b) and/or resting for 10-30 min, wherein the obtained mixture is characterized as flocculated; and
(d) addition of material C and material D as its solid powder followed by mixing and/or resting, wherein the obtained mixture is characterized as solidified.

6. The process for disinfection-flocculation-solidification as claimed in claim 5, wherein amount of the waste added is less than 1:1000 (v/v) of solution B for liquid waste and any immersible amount of solid waste or a mixture thereof.

7. The process for disinfection-flocculation-solidification as claimed in claim 5, wherein C is glacial acetic acid and the amount of C added is 0.1-3 mL per mL of the total aqueous mixture obtained in step (b).

8. The process for disinfection-flocculation-solidification as claimed in claim 5, wherein solid A is added at a minimum of 1% (w/v) and a maximum of 500% (w/v) of the total aqueous volume in the mixture obtained in step (c).

9. The process for disinfection-flocculation-solidification as claimed in claim 5, wherein the biomedical waste samples used in step (b) is selected from the group consisting of salt, sugar, metal salts and complexes, aqueous waste, hospital chemicals such as iodine, saliva, urine, blood, or any solid sample, inter alia cotton, tissue paper, needle, syringes, or swabs alone or in combination thereof, whereby disinfection is effected by the high pH of solution A containing B.

10. The process for disinfection-flocculation-solidification as claimed in claim 5, wherein said process assists in either flocculation leading to soft flocculated solids when terminated at step (c) as claimed in claim 5 or solidification leading to hard solids when continued to step (d) as claimed in claim 5.

11. A disinfection-flocculation-solidification and disposal device filled with the disinfection composition comprising four chemical components as claimed in claim 1, the device comprising:
(a) an upper container or compartment system;
(b) a second container or compartment system;
(c) a third container or compartment system;
(d) a bottom container or compartment system;
(e) a screw cap connected to the upper container or compartment system; and
(f) three breakable screw-caps, one connected between the upper and the second container or compartment systems, another connected between the second and the third container or compartment systems and a third connected to the third and the bottom container or compartment systems.

12. The disinfection-flocculation-solidification and disposal system as claimed in claim 11, wherein the upper container or compartment system is filled with solid powder of material D.

13. The disinfection-flocculation-solidification disposal system as claimed in claim 11, wherein the second container or compartment system is filled with solution C.

14. The disinfection-flocculation-solidification disposal system as claimed in claim 11, wherein the third container or compartment system is filled with the biomedical waste sample.

15. The disinfection-flocculation-solidification and disposal system as claimed in claim 11, wherein the bottom container or compartment system is filled with the aqueous solution of A mixed with B as claimed in claim 5.

16. The disinfection-solidification and disposal system as claimed in claim 11, wherein the biomedical sample is solid or liquid waste or their mixture.
